(19) United States
(12) Patent Application Publication
(10) Pub. No.: US 2024/0148561 A1
(43) Pub. Date: May 9, 2024

(54) CONTOURED FOAM DRESSING SHAPED FOR PROVIDING NEGATIVE PRESSURE TO INCISIONS IN THE BREAST

(71) Applicant: 3M Innovative Properties Company, St.Paul, MN (US)

(72) Inventors: Jonathan G. Rehbein, San Antonio, TX (US); Richard Kazala, San Antonio, TX (US); Enrique L. Sandoval, Fair Oaks Ranch, TX (US); Larry Tab Randolph, San Antonio, TX (US); Luke Perkins, San Antonio, TX (US); Ronald P. Silverman, Pikesville, MD (US)

(21) Appl. No.: 18/216,376

(22) Filed: Jun. 29, 2023

Related U.S. Application Data
(62) Division of application No. 16/156,387, filed on Oct. 10, 2018, now Pat. No. 11,730,631.
(60) Provisional application No. 62/578,173, filed on Oct. 27, 2017.

(51) Int. Cl. A61F 13/14; A61F 13/05; A61M 1/00

(52) U.S. Cl. CPC A61F 13/14; A61F 13/05; A61F 13/145; A61M 1/915; A61M 1/962; A61F 2013/00182; A61M 1/917

(57) ABSTRACT

A negative pressure wound dressing for use with breast incisions. The wound dressing includes a drape layer, a manifold layer, a base layer, and a reduced pressure interface. The drape layer has a first surface and a second, wound-facing, surface. The drape layer is substantially impermeable to liquid and substantially permeable to vapor. The manifold layer has a first surface and a second, wound-facing surface. The manifold layer has a perimeter defined by a first convex curved side surface defining a first lobe, a second convex curved side surface defining a second lobe, and a connecting portion between the first lobe and the second lobe. The base layer is configured to: (i) couple the drape layer to the manifold layer, and (ii) the dressing to a patient's tissue. The reduced pressure interface is integrated with the drape layer.

### CROSS-REFERENCE TO RELATED APPLICATIONS

The present application is a divisional filing of U.S. patent application Ser. No. 16/156,387, filed Oct. 10, 2018, entitled “CONTOURED FOAM DRESSING SHAPED FOR PROVIDING NEGATIVE PRESSURE TO INCISIONS IN THE BREAST,” which claims the benefit of and priority to U.S. Provisional Patent Application No. 62/578,173, filed Oct. 27, 2017, entitled “CONTOURED FOAM DRESSING SHAPED FOR PROVIDING NEGATIVE PRESSURE TO INCISIONS IN THE BREAST,” the entire contents of which are incorporated herein by reference.

### BACKGROUND

The present disclosure relates generally to a wound therapy system, and more particularly to a wound therapy system contoured to provide negative pressure wound therapy to the site of a breast incision.

Negative pressure wound therapy (NPWT) is a type of wound therapy that involves applying a negative pressure to a wound site to promote wound healing. NPWT can be used to treat wounds in the breast area caused by mastectomies, breast enhancement surgery, breast reconstruction surgery, or breast reduction surgery. Current negative pressure dressings used to treat the breast area are generally shaped in the form of a brassiere and include a first cup and a second cup made from a dressing material connected by a band of material. Such dressings must usually be provided in many sizes and, in the case of mastectomies, may require the use of a prosthetic device in one or both of the cups.

Recent developments in NPWT therapy include the use of adhesive wound dressings that can be positioned over a wound to treat the wound and the surrounding area. However, existing adhesive NPWT dressings are primarily linear dressings designed to treat linear wounds. In most instances, breast surgeries involve two generally perpendicular incisions. A first incision is a generally horizontal incision proximate a base of the breast and a second incision is generally perpendicular to the first incision and extends upward from the first incision and around a top of the nipple. The two incisions form an inverted T-shape. The existing NPWT dressings are configured to treat one of the first incision and the second incision, and must be customized to treat the specific incision pattern, breast size, and/or left breast or right breast of the patient.

### SUMMARY

One implementation of the present disclosure is a negative pressure wound therapy system for use with breast incisions. The system includes a wound dressing including a drape layer, a manifold layer, a reduced-pressure interface, a base layer, and an optional wound-interface layer. The drape layer is configured to provide a sealed space over a wound or incision, and has a first surface and a second, wound-facing, surface. The drape layer is substantially impermeable to liquid and substantially permeable to vapor. The manifold layer allows for the transmission of negative pressure to a patient's tissue, and has a first surface and a second, wound-facing surface. In some embodiments, the manifold layer is comprised of a hydrophobic foam, such as an open-cell polyurethane foam. The manifold layer has a perimeter defined by a first convex curved side surface defining a first lobe, a second convex curved side surface defining a second lobe, and a connecting portion between the first lobe and the second lobe. The reduced-pressure interface allows for the fluid communication of negative pressure from a negative pressure source into the dressing (through the drape layer), via a conduit configured to fluidly couple the negative pressure source and the reduced-pressure interface. The reduced-pressure interface is preferably integrated with the drape layer; though alternatively, it can be separate from the drape layer and configured to be coupled to the drape layer by a user. The base layer may comprise polyurethane film coated with an adhesive (such as acrylic or silicone adhesive) on both sides. The base layer may be (i) configured to secure the drape layer to the manifold layer and, if present, the wound-interface layer, and (ii) configured to secure the dressing to a patient's tissue. In some embodiments, the functionality of the base layer is provided by the drape layer, and a separate base layer is not included. In some embodiments, the wound-facing side of the base layer includes a hydrocolloid adhesive. The optional wound interface layer may comprise a wicking material, and may optionally include antimicrobials (such as silver).

Another implementation of the present disclosure is a negative pressure wound dressing for use with breast incisions. The wound dressing includes a drape layer, a manifold layer, a base layer, and a reduced pressure interface. The drape layer has a first surface and a second, wound-facing, surface. The drape layer is substantially impermeable to liquid and substantially permeable to vapor. The manifold layer has a first surface and a second, wound-facing surface. The manifold layer has a perimeter defined by a first convex curved side surface defining a first lobe, a second convex curved side surface defining a second lobe, and a connecting portion between the first lobe and the second lobe. The base layer is (i) configured to secure the drape layer to the manifold layer, and (ii) configured to secure the dressing to a patient's tissue. The reduced pressure interface is integrated with the drape layer.

Another implementation of the present disclosure is a wound dressing for negative pressure wound therapy treatment of breast incisions including a manifold layer and a drape layer that is substantially impermeable to liquid and substantially permeable to vapor. The manifold layer includes a first surface, a second, wound-facing, surface, and a plurality of scores formed in the first surface and extending towards the second surface. The plurality of scores define a geometric scoring pattern. The manifold layer is bendable about the plurality of scores.

Another implementation of the present disclosure is a negative pressure wound dressing for use with breast incisions. The wound dressing includes a drape layer, a manifold layer, a base layer, and a reduced-pressure interface. The drape layer has a first surface and a second, wound-facing, surface. The drape layer is substantially impermeable to liquid and substantially permeable to vapor. The manifold layer has a first surface and a second, wound-facing surface. The manifold layer includes a perimeter defined by a first curved corner having first radius of curvature, a second curved corner having a second radius of curvature, and a third curved corner having a third radius of curvature. The third radius of curvature is smaller than the first radius of curvature and the second radius of curvature. The base layer is (i) configured to secure the drape layer to the manifold layer and (ii) configured to secure the wound dressing to a patient's tissue. The reduced-pressure interface is integrated with the drape layer.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a front view of a wound dressing according to an exemplary embodiment.

FIG. 2 is a perspective view of the wound dressing of FIG. 1 according to an exemplary embodiment.

FIG. 3 is an exploded view of the wound dressing of FIG. 1 according to an exemplary embodiment.

FIG. 4 is a side cross-sectional view of the wound dressing of FIG. 1 adhered to a patient according to an exemplary embodiment.

FIG. 5 is a perspective view of a manifold layer of the wound dressing of FIG. 1 according to an exemplary embodiment.

FIG. 6 is an exploded view of a manifold layer of the wound dressing of FIG. 1 according to another exemplary embodiment.

FIG. 7 is a perspective view of a manifold layer of the wound dressing of FIG. 1 according to another exemplary embodiment.

FIG. 8 is a perspective view of a manifold layer of the wound according to another exemplary embodiment.

FIG. 9 is a top view of the wound dressing of FIG. 1 aligned with a representative person's torso.

FIG. 10 is a perspective view of the wound dressing of FIG. 1 adhered to a representative person's torso.

FIG. 11 is a front view of a wound dressing according to an exemplary embodiment.

### DETAILED DESCRIPTION

### Overview

Referring generally to the FIGURES, a wound therapy system for treating wounds of curved body parts is shown, according to various embodiments. More specifically, the wound therapy system is for treating wounds in the breast area, although the wound therapy system can also be deployed on other curved parts of the body, such as the buttocks, harvest sites for skin grafting (e.g. the back of a leg), and the lower back. The wound therapy system includes a wound dressing and a negative pressure wound therapy (NPWT) system. The phrase “negative pressure” means a pressure less than an ambient or atmospheric pressure. While the amount and nature of reduced pressure applied to the wound site can vary according to the application, the reduced pressure typically is between −5 mm Hg and −500 mm Hg and more typically between −100 mm Hg and −300 mm Hg. The wound dressing described herein is shaped to cover the entire breast area (e.g. extend between a side of the patient's rib cage generally beneath the armpit near the lymph node gland to the sternum region and an upper portion of the chest area). In some embodiments, the profile of the wound dressing is generally heart-shaped and includes a curved, slightly concave portion positionable proximate a patient's armpit, a first lobe (e.g. slightly convex) for covering an upper portion of the breast and a second lobe (e.g. slightly convex) for covering a bottom portion of the breast, curving around the ribcage, and extending beneath the armpit. In some embodiments, the profile of the wound dressing is generally shaped like a guitar pick (e.g., generally triangular and having curved sides and corners). The wound dressing described herein can be configured to conform to applications involving two-dimensional and/or three-dimensional contours. For example, when the wound dressing is used to treat a full and/or partial mastectomy, the dressing is configured to conform to a side of the patient's ribcage and a generally flat (e.g. two-dimensional) front portion of the patient's rib cage. In instances where the wound dressing is used to treat a partial mastectomy, a breast enhancement incision, or a breast reduction incision, the wound dressing is configured to conform to a side of the patient's ribcage and a curved (e.g. a three-dimensional) contour formed defined by the breast and the front portion of the ribcage. The profile shape of the wound dressing is generally symmetric to allow placement of the wound dressing on either the left or the right breast.

In the illustrated embodiments, the manifold layer of the wound dressing includes a scoring pattern to allow the manifold layer to bend to conform to three-dimensional curved shapes. The scores extend generally to a partial depth of the thickness (e.g. 7 mm, etc.) into the manifold layer. In some embodiments, the scoring pattern is generally hexagonal. In other embodiments, the scoring pattern is generally quadrilateral. For example, the scores may form squares, parallelograms, or rectangles. In other embodiments, the scoring pattern is concentric scoring following a shape of a perimeter of the manifold layer. The manifold layer is configured to wick fluid (e.g. exudate) from the wound and includes in-molded manifold layer structures for distributing negative pressure throughout the wound dressing during negative pressure wound therapy treatments.

In some embodiments, a portion of the scores in a central area of the manifold layer of the wound include perforations that extend through a width of the manifold layer. The perforations permit selective removal of one or more pieces of the manifold layer to allow visualization of the nipple when the wound dressing is secured to a patient. Visualization of the nipple is intended to allow a healthcare practitioner to observe the health of the wound while leaving the dressing intact.

The wound therapy system may include a removed-fluid container and a pump. The removed-fluid container can be configured to store a fluid removed from the wound site (e.g., wound exudate, etc.). The removed-fluid container can be fluidly coupled to the wound site via a fluid removal line. The NPWT can help reduce the chance of the wounds developing seroma, scaring, infection, or other adverse complications.

In some embodiments, when two wound dressings are used on the same patient, the two wound dressings can be connected using a Y-connection so that the same pump and removed-fluid container can be used to treat the wounds. The pump and/or the Y-connection can include at least one valve so that different amounts of negative pressure can be exerted on each breast if desired.

Additional features and advantages of the wound therapy system are described in detail below.

### Wound Dressing

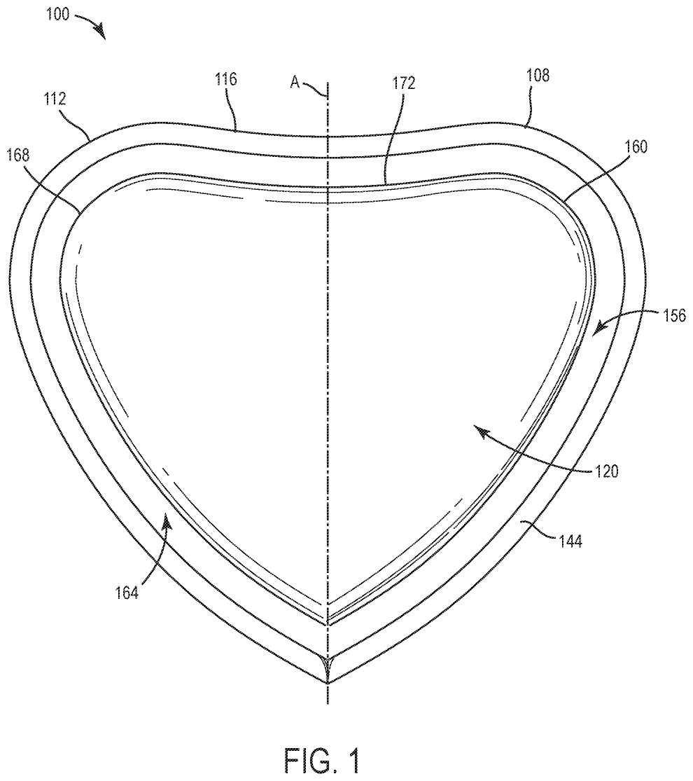

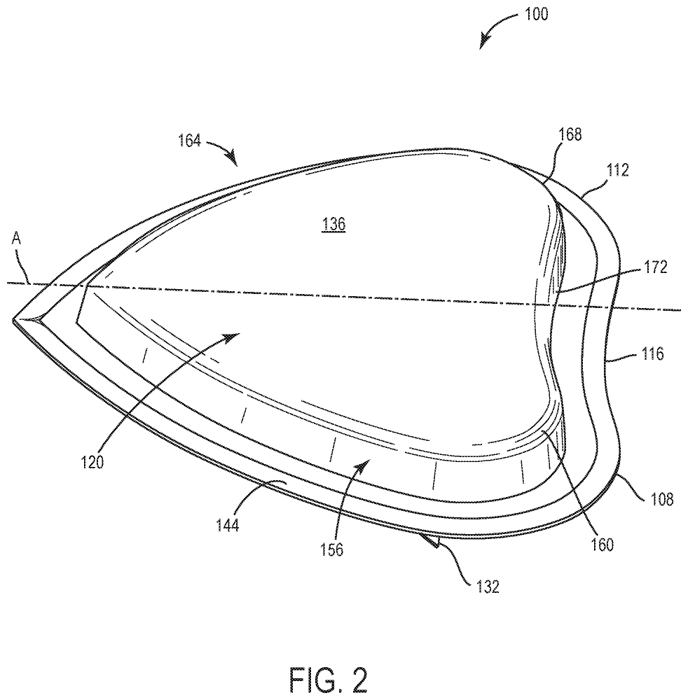

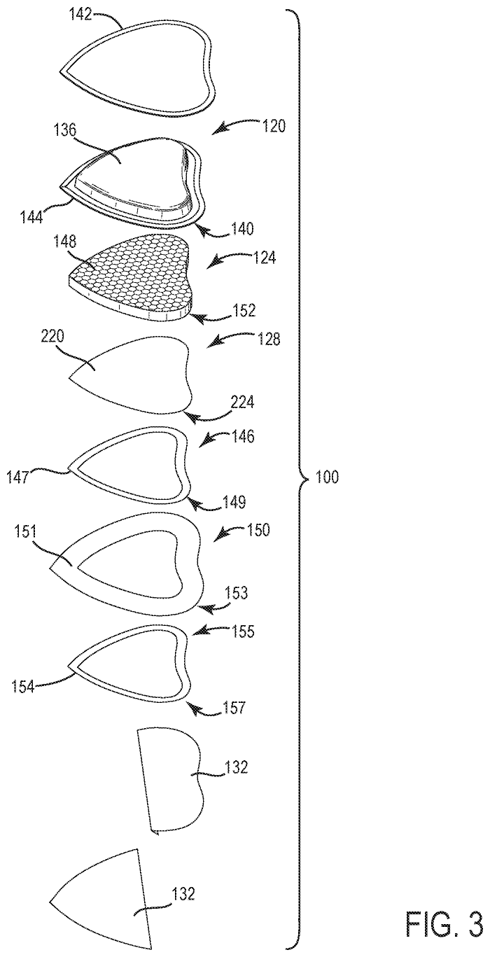

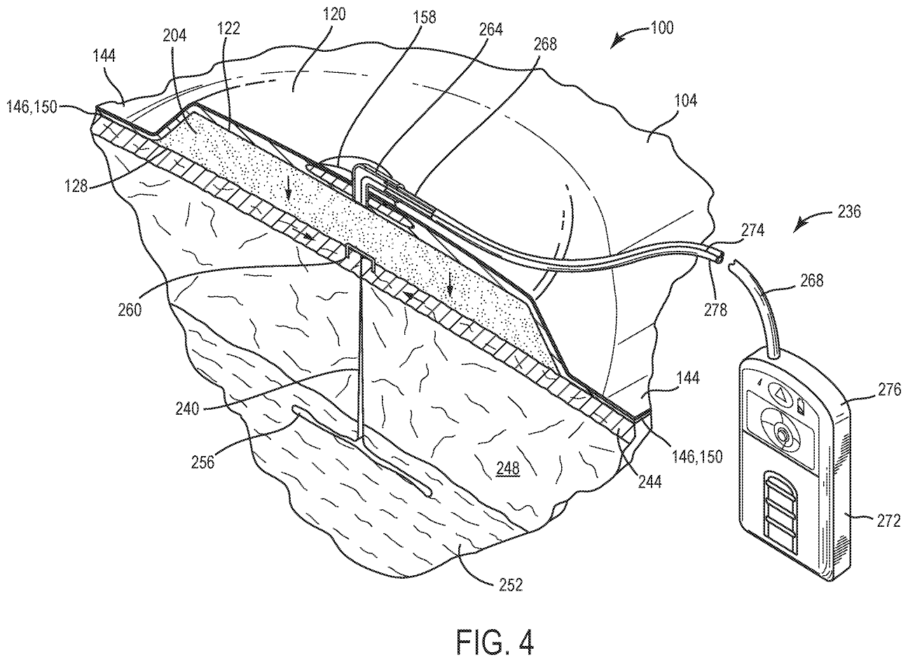

Referring now to FIGS. 1-4, a wound dressing 100 is shown, according to an exemplary embodiment. FIG. 1 is a front view of the wound dressing 100. FIG. 2 is a perspective view of the wound dressing 100. FIG. 3 is an exploded view illustrating several layers 120-154 of the wound dressing 100. FIG. 4 is a cross-sectional view of the wound dressing 100 adhered to a surface 104, such as a patient's torso.

In various embodiments, the wound dressing 100 can be formed as a substantially flat sheet for topical application to wounds. The wound dressing 100 can lie flat for treatment of substantially flat wounds and is also configured to bend to conform to body surfaces having high curvature, such as breasts. The wound dressing 100 has a profile or a perimeter that is generally heart-shaped and includes a first lobe 108 (e.g. convex portion) and a second lobe 112 (e.g. convex portion) that define a concave portion 116 therebetween. The wound dressing 100 is generally symmetric about an axis A. It is contemplated that the size of the wound dressing can range from 180 cm² to 1000 cm². More preferably, the size of the wound dressing can range from 370 cm² to 380 cm², 485 cm² to 495 cm², and/or 720 cm² to 740 cm². However, other shapes and sizes of wound dressing 100 are also possible depending on the intended use. For example, for some uses, the wound dressing 100 may have asymmetrically-shaped lobes 108, 112.

The wound dressing 100 is shown to include a plurality of layers, including a drape layer 120, a manifold layer 124, a wound-interface layer 128, a rigid support layer 142, a first adhesive layer 146, a second adhesive layer 150, and a patient-contacting layer 154. In some embodiments, the wound dressing 100 includes a removable cover sheet 132 to cover the manifold layer 124, the wound-interface layer 128, the second adhesive layer 150, and/or the patient-contacting layer 154 before use.

### Drape Layer

The drape layer 120 is shown to include a first surface 136 and a second, wound-facing, surface 140 opposite the first surface 136. When the wound dressing 100 is applied to a wound, the first surface 136 faces away from the wound, whereas the second surface 140 faces toward the wound. The drape layer 120 supports the manifold layer 124 and the wound-interface layer 128 and provides a barrier to passage of microorganisms through the wound dressing 100. The drape layer 120 is configured to provide a sealed space over a wound or incision. In some embodiments, the drape layer 120 is an elastomeric material or may be any material that provides a fluid seal. “Fluid seal” means a seal adequate to hold pressure at a desired site given the particular reduced-pressure subsystem involved. The term “elastomeric” means having the properties of an elastomer and generally refers to a polymeric material that has rubber-like properties. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. As non-limiting examples, the drape layer 120 may be formed from materials that include a silicone, 3M Tegaderm® drape material, acrylic drape material such as one available from Avery, or an incise drape material.

The drape layer 120 may be substantially impermeable to liquid and substantially permeable to water vapor. In other words, the drape layer 120 may be permeable to water vapor, but not permeable to liquid water or wound exudate. This increases the total fluid handling capacity (TFHC) of wound dressing 100 while promoting a moist wound environment. In some embodiments, the drape layer 120 is also impermeable to bacteria and other microorganisms. In some embodiments, the drape layer 120 is configured to wick moisture from the manifold layer 124 and distribute the moisture across the first surface 136.

In the illustrated embodiment, the drape layer 120 defines a cavity 122 (FIG. 4) for receiving the manifold layer 124, the wound-interface layer 128, and the first adhesive layer 146. As shown in FIG. 2, the manifold layer 124, the wound-interface layer 128, and the first adhesive layer 146 can have a similar perimeter or profile. In some embodiments, a perimeter of the drape layer 120 extends beyond (e.g. circumscribes) the perimeter of the manifold layer 124 to provide a margin 144. The first adhesive layer 146 includes a first surface 147 and a second, wound-facing surface 149. Both first surface 147 and the second surface 149 are coated with an adhesive, such as an acrylic adhesive, a silicone adhesive, and/or other adhesives. The first surface 147 of the first adhesive layer 146 is secured to the second surface 224 of the wound-interface layer 128. The second surface 149 of the first adhesive layer 146 is secured to the second adhesive layer 150. The second adhesive layer 150 includes a first surface 151 and a second, wound-facing surface 153. The second surface 149 of the first adhesive layer 146 is secured to the first surface 151 of the second adhesive layer 150. The second surface 153 of the second adhesive layer 150 is coated with an acrylic adhesive, a silicone adhesive, and/or other adhesives. The adhesive applied to the second surface 153 of the second adhesive layer 150 is intended to ensure that the wound dressing 100 adheres to the surface 104 of the patient's skin (as shown in FIGS. 4, 9, and 10) and that the wound dressing 100 remains in place throughout the wear time. The second adhesive layer 150 has a perimeter or profile that is similar to a perimeter or profile of the margin 144. In the illustrated embodiment, the first surface 151 of the second adhesive layer 150 is welded to the margin 144. In other embodiments, the first surface 151 of the second adhesive layer is secured to the margin 144 using an adhesive, such as an acrylic adhesive, a silicone adhesive, or another type of adhesive. The patient-contacting layer 154 includes a first surface 155 and a second, wound-facing surface 157. In some embodiments, the patient-contacting layer 154 can be made of a hydrocolloid material, a silicone material or another similar material. The first surface 155 of the patient-contacting layer 154 can be secured to the second adhesive layer 150. The patient-contacting layer 154 follows a perimeter of the manifold layer 124. In some embodiments, the patient-contacting layer 154 can be made of a polyurethane film coated with an acrylic or silicone adhesive on both surfaces 155, 157. In some embodiments, the patient-contacting layer 154 can include a hydrocolloid adhesive on the second, wound-facing, surface 157. The margin 144 and/or the second adhesive layer 150 may extend around all sides of the manifold layer 124 such that the wound dressing 100 is a so-called island dressing. In other embodiments, the margin 144 and/or the second adhesive layer 150 can be eliminated and the wound dressing 100 can be adhered to the surface 104 using other techniques. In some embodiments, the first adhesive layer 146, the second adhesive layer 150, and the patient-contacting layer 154 can collectively form a base layer that includes an adhesive on both sides that is (i) configured to secure the drape layer 120 to the manifold layer 124, the optional wound-interface layer 128, and (ii) configured to secure the wound dressing 100 to a patient's tissue. In some embodiments, the base layer can be integrally formed with the drape layer 120. In some embodiments, the base layer can be a layer of a polyurethane film having a first surface and a second, wound-facing surface. Both the first surface and the second surface can be coated with an adhesive (such as an acrylic or silicone adhesive). In some embodiments, the wound-facing surface of the base layer can include a hydrocolloid adhesive.

In some embodiments, a reduced-pressure interface 158 can be integrated with the drape layer 120. The reduced-pressure interface 158 can be in fluid communication with the negative pressure system through a removed fluid conduit 268 (FIG. 4). The reduced-pressure interface 158 is configured to allow fluid communication between a negative pressure source and the wound dressing 100 (e.g., through the drape layer 120) via a removed fluid conduit coupled between the reduced-pressure interface 158 and the negative pressure source such that negative pressure generated by the negative pressure source can be applied to the wound dressing 100 (e.g., through the drape layer 120). In some embodiments, the reduced-pressure interface 158 can be integrated (e.g., integrally formed) with the drape layer 120. In other embodiments, the reduced-pressure interface 158 can be separate from the drape layer 120 and configured to be coupled to the drape layer 120 by a user.

With continued reference to FIG. 2, the rigid support layer 142 is positioned above the first surface 136 of the drape layer 120. The rigid support layer 142 is spaced from but proximate the margin 144 and the second adhesive layer 150. The rigid support layer 142 is made of a rigid material and helps the wound dressing 100 maintain rigidity before the wound dressing 100 is secured to the surface 104 of the patient. The rigid support layer 142 is intended to be removed from the drape layer 120 after the wound dressing 100 has been secured to the surface 104 of the patient.

In some embodiments, the second surface 140 of the drape layer 120 contacts the manifold layer 124. The second surface 140 of the drape layer 120 may be adhered to the manifold layer 124 or may simply contact the manifold layer 124 without the use of an adhesive.

In some embodiments, the adhesive applied to the second surface 140 of the drape layer 120 is moisture vapor transmitting and/or patterned to allow passage of water vapor therethrough. The adhesive may include a continuous moisture vapor transmitting, pressure-sensitive adhesive layer of the type conventionally used for island-type wound dressings (e.g. a polyurethane-based pressure sensitive adhesive).

### Manifold Layer

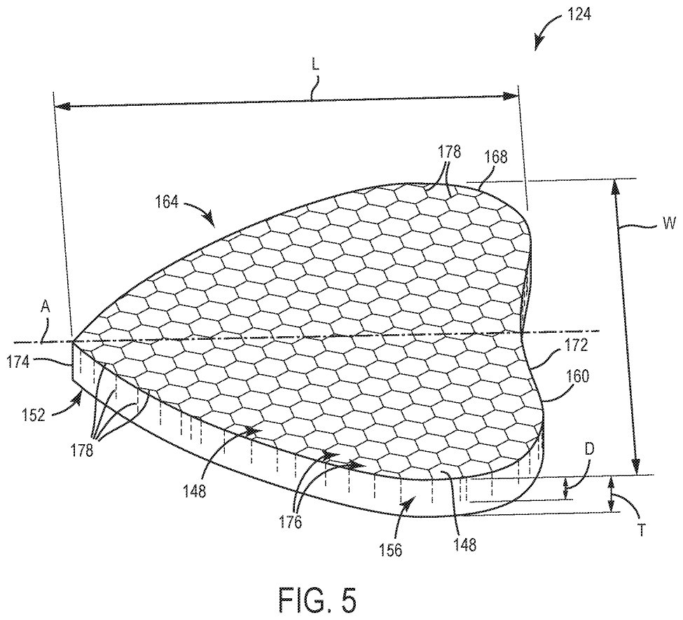

Referring to FIG. 5, the manifold layer 124 is shown to include a first surface 148 and a second, wound-facing surface 152 opposite the first surface 148. When the wound dressing 100 is applied to a wound, the first surface 148 faces away from the wound, whereas the second surface 152 faces toward the wound. In some embodiments, the first surface 148 of the manifold layer 124 contacts the second surface 140 of the drape layer 120. In some embodiments, the second surface 152 of the manifold layer 124 contacts the wound-interface layer 128. The manifold layer 124 is configured for transmission of negative pressure to the patient's tissue at and/or proximate a wound and/or incision. The manifold layer 124 is configured to wick fluid (e.g. exudate) from the wound and includes in-molded manifold layer structures for distributing negative pressure throughout the wound dressing 100 during negative pressure wound therapy treatments.

The manifold layer 124 can be made from a porous and permeable foam-like material and, more particularly, a reticulated, open-cell polyurethane or polyether foam that allows good permeability of wound fluids while under a reduced pressure. One such foam material that has been used is the V.A.C.® Granufoam™ material that is available from Kinetic Concepts, Inc. (KCl) of San Antonio, Tex. Any material or combination of materials might be used for the manifold layer 124 provided that the manifold layer 124 is operable to distribute the reduced pressure and provide a distributed compressive force along the wound site.

The reticulated pores of the Granufoam™ material that are in the range from about 400 to 600 microns, are preferred, but other materials may be used. The density of the manifold layer material, e.g., Granufoam™ material, is typically in the range of about 1.3 lb/ft³-1.6 lb/ft³ (20.8 kg/m 3-25.6 kg/m 3). A material with a higher density (smaller pore size) than Granufoam™ material may be desirable in some situations. For example, the Granufoam™ material or similar material with a density greater than 1.6 lb/ft³ (25.6 kg/m 3) may be used. As another example, the Granufoam™ material or similar material with a density greater than 2.0 lb/ft³ (32 kg/m 3) or 5.0 lb/ft³ (80.1 kg/m 3) or even more may be used. The more dense the material is, the higher compressive force that may be generated for a given reduced pressure. If a foam with a density less than the tissue at the tissue site is used as the manifold layer material, a lifting force may be developed. In one illustrative embodiment, a portion, e.g., the edges, of the wound dressing 100 may exert a compressive force while another portion, e.g., a central portion, may provide a lifting force.

The manifold layer material may be a reticulated foam that is later felted to thickness of about one third (¼) of the foam's original thickness. Among the many possible manifold layer materials, the following may be used: Granufoam™ material or a Foamex® technical foam (www.foamex.com). In some instances it may be desirable to add ionic silver to the foam in a microbonding process or to add other substances to the manifold layer material such as antimicrobial agents. The manifold layer material may be isotropic or anisotropic depending on the exact orientation of the compressive forces that are desired during the application of reduced pressure. The manifold layer material may also be a bio-absorbable material.

As shown in FIGS. 1-3 and 5, the manifold layer 124 is generally symmetrical, heart-shaped, and includes a first convex curved side 156 defining a first lobe 160, a second convex curved side 164 defining a second lobe 168, and a concave connecting portion 172 extending therebetween. The manifold layer 124 can have a width W ranging between 8 cm and 33 cm, and more preferably between 17 cm and 33 cm. The manifold layer 124 can have a length L ranging between 7 cm and 35 cm, and more preferably between 14 cm and 30 cm. The manifold layer 124 can have a thickness T ranging between 14 mm and 24 mm, and more preferably 19 mm. The first lobe 160 and the second lobe 168 are convex and can have a radius of curvature ranging between 3 cm and 10 cm, and more preferably from 5 cm to 9 cm. The connecting portion 172 is generally concave and can have a radius of curvature ranging between 20 cm and 33 cm, and more preferably from 22 cm to 28 cm. The first curved side 156 and the second curved side 164 form a point 174 positioned generally opposite the connecting portion 172. In the illustrated embodiment, the first curved side 156 and the second curved side 164 are generally symmetric about the axis A.

As is best shown in FIG. 5, a scoring pattern 176 is formed in the first surface 148 of the manifold layer 124. The scoring pattern 176 is shown for example as an arrangement of “slits” or scores (e.g., “mango-cuts”) formed in the manifold layer 124 (e.g. formed by laser-scoring or other suitable processes). More particularly, the scoring pattern 176 is cut into the first surface 148 of the manifold layer 124. In the embodiment of FIG. 5, the scoring pattern 176 extends between the first surface 148 and the second surface 152 but does not extend completely to the second surface 152. The scoring pattern 176 can have a depth D that ranges between 5 mm and 12 mm, and more preferably is approximately 7 mm. According to the illustrated embodiment, the scoring pattern 176 is a generally hexagonal pattern. However, in other embodiments, the scoring pattern 176 can be a different geometrical pattern. When the wound dressing 100 is used on a generally flat (e.g. two-dimensional) surface 104 or portion of a surface 104, such as for example a front of a patient's ribcage after a mastectomy or a side of the patient's ribcage, the scores 178 of the scoring pattern 176 are generally vertical and are in close proximity to adjacent scores 178 of the scoring pattern 176. In instances when the wound dressing 100 is secured to a curved (e.g. three-dimensional) surface, such as a breast or a transition portion of the surface that extends between the side of the ribcage and the front of the ribcage, the scores 178 of the scoring pattern 176 splay apart to facilitate bending of the manifold layer 124 so that the manifold layer 124 closely conforms to the shape of the curved surface 104. The scoring pattern 176 allows the manifold layer 124 to conform to both substantially flat surfaces and to a wide range of curved surfaces 104. The hexagonal scoring pattern 176 facilitates conforming to highly curved surfaces 104 by providing six scores 178 about which the manifold layer 124 can bend (e.g. the manifold layer 124 can bend in six different directions proximate each hexagon cut into the first surface 148). In implementations where the surface 104 is the breast or is proximate the breast of a patient, the scoring pattern 176 allows a single size of manifold layer 124 to conform to varying sizes of breasts.

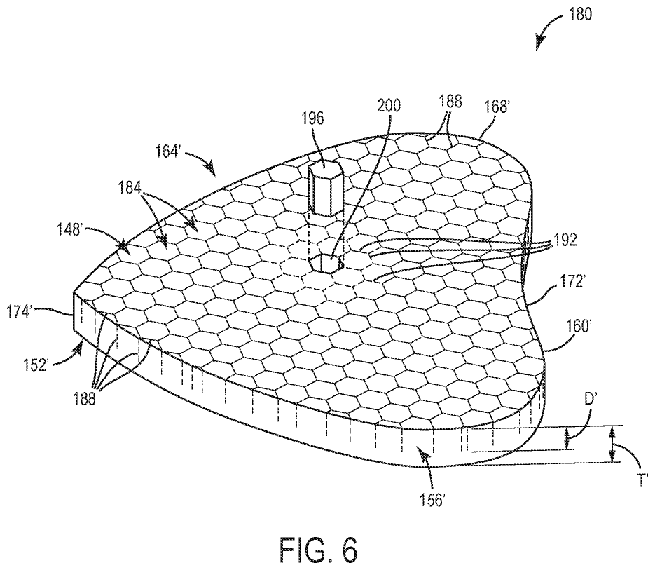

FIG. 6 illustrates a manifold layer 180 according to another embodiment. The manifold layer 180 is generally similar to the manifold layer 124. The manifold layer 180 can be incorporated into the wound dressing 100 as described above with respect to the manifold layer 124. Like numbers are indicated by the same number and parts of the manifold layer 180 are indicated using the prime symbol “‘ ”.

The manifold layer 180 includes a scoring pattern 184 formed in the first surface 148’. The scoring pattern 184 is shown for example as an arrangement of “slits” or scores (e.g., “mango-cuts”) formed in the manifold layer 180 (e.g. formed by laser-scoring or other suitable processes). Preferably, the scoring pattern 184 is cut into the first surface 148′. In the embodiment shown in FIG. 6, the scoring pattern 184 is a hexagonal pattern. In other embodiments, the scoring pattern 184 can be a different geometrical pattern. The scoring pattern 184 includes a first portion 188 of scores proximate a perimeter of the manifold layer 180 and a second portion 192 of scores proximate a center of the manifold layer 180. The first portion 188 of scores, which are shown in phantom in FIG. 6, extends from the first surface 148′ toward the second surface 152′, but does not penetrate the second surface 152′. The first portion 188 of scores can have a depth D′ that ranges between 5 mm and 12 mm, and more preferably is approximately 7 mm. The second portion 192 of scores, which is indicated by the dashed lines in FIG. 6, include a cut portion and a perforated portion. The perforations of the perforated portion penetrate through to the second surface 152′. The perforations facilitate removal of at least one piece 196 of the second portion 192 of the manifold layer 124. FIG. 6 illustrates a piece 196 of the manifold layer 180 that has been removed to form a through-opening 200 in the manifold layer 180. The through-opening 200 allows for visualization of the wound through the manifold layer 180 when the wound dressing 100 is secured to the patient. More particularly, when the manifold layer 180 is used to treat wounds in the breast area, the second portion 192 is positioned to cover a center of the breast so that at least one piece 196 of the second portion 192 can be selectively removed to allow visualization of the nipple. Visual inspection of the color of the nipple is generally understood to be indicative of the health and/or condition of the wounds in the breast area while the dressing remains intact and without interrupting the negative pressure therapy of the wound. To facilitate viewing of the wound through the wound dressing 100, the drape layer 120 is preferably transparent in embodiments of the wound dressing 100 that include the manifold layer 180.

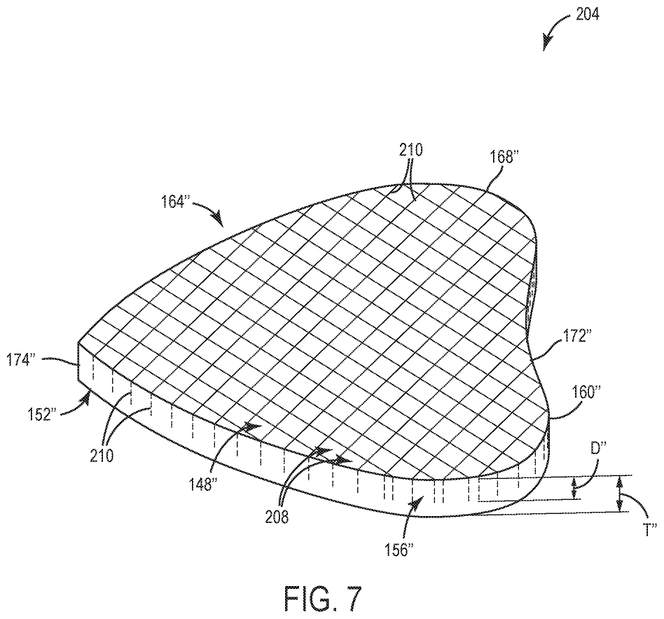

FIG. 7 illustrates a manifold layer 204 according to another embodiment. The manifold layer 204 is generally similar to the manifold layer 124. The manifold layer 204 can be incorporated into the wound dressing 100 as described above with respect to the manifold layer 124. Like numbers are indicated by the same number and parts of the manifold layer 204 are indicated using the double prime symbol “ ” “.

The manifold layer 204 includes a scoring pattern 208 formed in the first surface 148”. The scoring pattern 208 is shown for example as an arrangement of “slits” or scores (e.g., “mango-cuts”) formed in the manifold layer 204 (e.g. formed by laser-scoring or other suitable processes). More particularly, the scoring pattern 208 is cut into the first surface 148″. In the embodiment shown in FIG. 7, the scoring pattern 208 is a generally square pattern. In other embodiments, the scoring pattern 208 can be a different geometrical shape, for example, rectangular, parallelogram, diamond, rhombus, or any other quadrilateral shape. In the embodiment of FIG. 7, the scoring pattern 208 extends between the first surface 148″ and the second surface 152″ but does not extend completely through to the second surface 152″. The scoring pattern 208 can have a depth D″ that ranges between 5 mm and 12 mm, and more preferably is 7 mm. In other embodiments, the scoring pattern 208 can facilitate removal of at least some of a center portion of the manifold layer 204 as described above with respect to FIG. 6. When the wound dressing 100 is used on a generally flat (e.g. two-dimensional) surface 104 or portion of a surface 104, such as for example a front of a patient's ribcage after a mastectomy or a side of the patient's ribcage, the scores 210 of the scoring pattern 208 are generally vertical and are in close proximity to adjacent scores 210. In instances when the wound dressing 100 is secured to a curved (e.g. three-dimensional) surface 104, such as a breast or a transition portion of the surface 104 that extends between the side of the ribcage and the front of the ribcage, the scores 210 of the scoring pattern 208 splay apart to facilitate bending of the manifold layer 204 so that the manifold layer 204 closely conforms to the shape of the curved surface 104. The scoring pattern 208 allows the manifold layer 204 to conform to both substantially flat surfaces and to a wide range of curved surfaces 104. The square scoring pattern 208 facilitates conforming to curved surfaces 104 by providing four scores 210 about which the manifold layer 204 can bend (e.g. the manifold layer can bend in four different directions proximate each square cut into the first surface 148″). In implementations where the surface 104 is the breast or is proximate the breast of a patient, the scoring pattern 208 allows a piece of manifold layer 204 to conform to varying sizes of breasts.

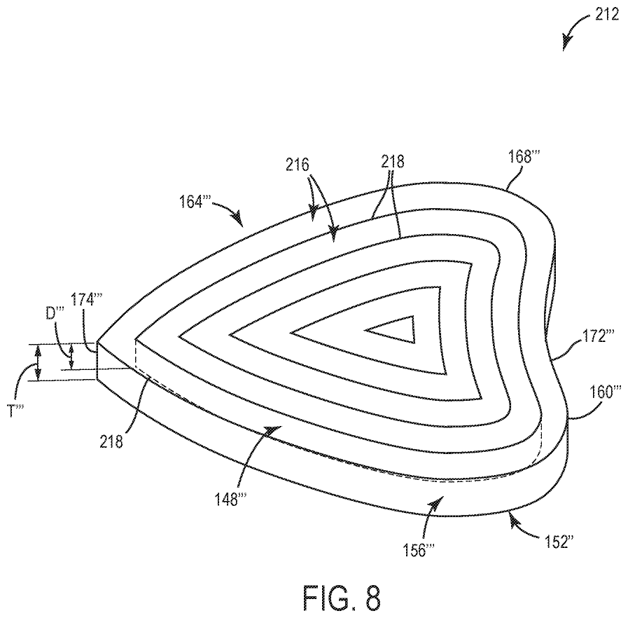

FIG. 8 illustrates a manifold layer 212 according to a different embodiment. The manifold layer 212 is generally similar to the manifold layer 124. The manifold layer 212 can be incorporated into the wound dressing 100 as described above with respect to the manifold layer 124. Like numbers are indicated by the same number and parts of the manifold layer 212 are indicated using the triple prime symbol “‘” “.

The manifold layer 212 includes a scoring pattern 216 formed in the first surface 148”’. The scoring pattern is shown for example as an arrangement of “slits” or scores formed in the manifold layer 212 (e.g. formed by laser-scoring or other suitable processes). Preferably, the scoring pattern 216 is cut into the first surface 148′″. In the embodiment shown in FIG. 8, the scoring pattern 216 includes concentric scores 218 in a shape of a perimeter of the manifold layer 212. In the embodiment of FIG. 8, the scoring pattern 216 extends between the first surface 148′″ and the second surface 152′″ but does not extend completely through to the second surface 152′″. The scoring pattern 216 can have a depth D′″ that ranges between 5 mm and 12 mm, and more preferably is approximately 7 mm. In instances where the wound dressing 100 is used on a generally flat (e.g. two-dimensional) surface 104 or portion of a surface 104, such as for example a front of a patient's ribcage after a mastectomy or a side of the patient's ribcage, the scores of the scoring pattern 216 are generally vertical and are in close proximity to adjacent scores. In instances when the wound dressing 100 is secured to a curved (e.g. three-dimensional) surface 104, such as a breast or a transition portion of the surface 104 that extends between the side of the ribcage and the front of the ribcage, the scores 218 of the scoring pattern 216 splay apart to facilitate bending of the manifold layer 212 so that the manifold layer 212 closely conform to the shape of the curved surface 104. The scoring pattern 216 allows the manifold layer 212 to conform to both substantially flat surfaces and a wide range of curved surfaces 104. In implementations where the surface 104 is or is proximate the breast area of a patient, the scoring pattern 216 allows a single size of manifold layer 212 to conform to varying sizes of breasts.

Some embodiments may include manifold layers having a first geometric pattern and a second geometric pattern different than the first geometric pattern. In such embodiments, the first geometric pattern may have more sides than the second geometric pattern, and thus be able to conform to a more highly curved shape. In such an embodiment, the first geometric pattern may be positioned proximate a center of the manifold layer and the second geometric pattern may be positioned proximate a perimeter of the manifold layer.

### Wound-Interface Layer

The wound-interface layer 128 is shown to include a first surface 220 and a second, wound-facing surface 224 opposite the first surface 220. When the wound dressing 100 is applied to the wound, the first surface 220 faces away from the wound, whereas the second surface 152 faces toward the wound. In some embodiments, the first surface 220 of the wound-interface layer 128 contacts the second surface 224 of the manifold layer 124. In some embodiments, the second surface 224 of the wound-interface layer 128 contacts the surface 104 of the patient. In some embodiments, the wound dressing 100 may not include the wound-interface layer 128.

The wound-interface layer 128 is made of a wicking material that is fluid-permeable and intended to not irritate the patient's skin. In the illustrated embodiment, the wound-interface layer is a polyester pique-knit fabric, such as Milliken Fabric. In other embodiments, other permeable and non-irritating fabrics can be used. The wound-interface layer 128 can also be treated with antimicrobial materials. In the illustrated embodiment, the wound-interface layer 128 includes silver ions as an antimicrobial material. Other anti-microbial materials may be used in other embodiments.

### Deployment of the Dressing

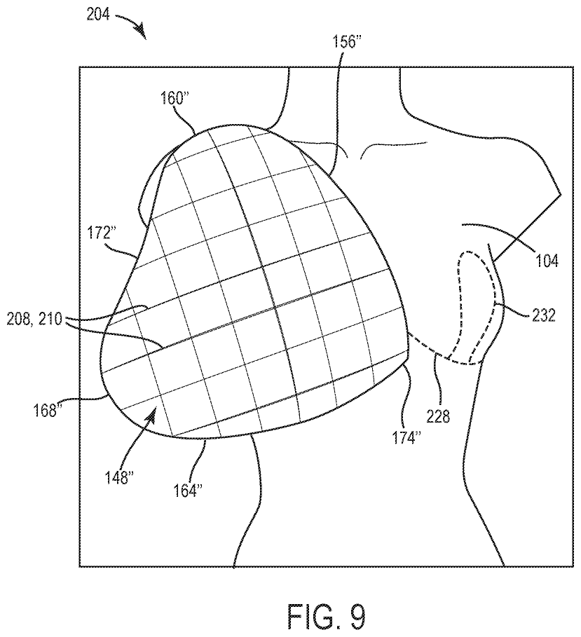

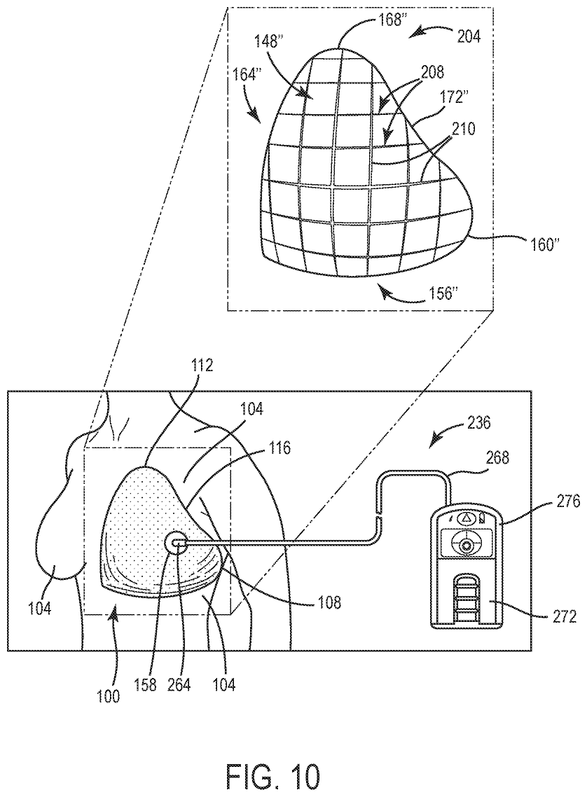

FIG. 9 illustrates the manifold layer 204 positioned proximate a representative model of a woman's torso. While the manifold layer 204 is shown in FIGS. 9 and 10, the manifold layers 124, 180, and 212 can be deployed in a similar manner. Exemplary intersecting incisions used in breast surgeries such as full and/or partial mastectomies, breast enhancements, and/or breast reductions are shown on a left breast of the model. Although FIG. 9 illustrates the manifold layer 204, the wound dressing 100 is positioned on the patient in a similar manner. A first incision 228 is positioned proximate the bottom (e.g. towards the feet) of the breast. A second incision 232 extends upward (e.g. towards the head) from the first incision 228, surrounds the nipple, and extends downward toward the first incision 228. Such intersecting incisions are typically referred to as T-shaped incisions. Breast surgeries may include further incisions proximate the armpit and/or the lymph node proximate the armpit (not shown). As shown in FIG. 9, the manifold layer 204 (and the wound dressing 100) is sized to cover the surface including the entire breast area, including the first incision 228, the second incision 232, and any incisions proximate the armpit. A further advantage of covering the entire breast area is that the manifold layer 204 (and the wound dressing 100) can provide support to the whole breast area during the negative pressure therapy. In some embodiments, the wound dressing 100 can be used with topically applied pharmaceutical compounds. For example, the wound dressing 100 can be used in conjunction with a silicone gel applied proximate the first incision 228 and the second incision 232. The silicone gel can reduce scarring at or near the incisions 228, 232. In another example, the wound dressing 100 can be used in conjunction with a nitroglycerin ointment applied at or near the nipple area. The nitroglycerin ointment can increase perfusion at or near the nipple area.

FIG. 9 illustrates by way of example the manifold layer 204 positioned above a surface 104 defined by the right breast for treatment of the right breast area. When the manifold layer 204 is used to support the right breast, the manifold layer 204 is positioned so that the connecting portion 172″ is positioned proximate the right armpit. The first lobe 160″ covers an upper portion of the breast area. The second lobe 168″ covers a bottom portion of the breast area and can be curved around the body to cover a portion of a right side of the torso. The point 174″ is positioned proximate a bottom of the sternum. By comparison with the incisions 228, 232 shown on the left breast, both the first incision 228 and the second incision 232 are positioned beneath the manifold layer 204 shown in FIG. 9. The manifold layer 204 has not yet been curved to conform to the breast, so the scores of the scoring pattern 208 are narrow.

FIG. 10 illustrates the wound dressing 100 secured to a surface defined by the woman's left breast. The inset shows the manifold layer 204. The first lobe 160″ covers a lower portion of the breast area and is curved around the ribcage to cover a portion of a left side of the torso proximate the left armpit. The second lobe 168″ covers an upper portion of the breast area. The manifold layer 204 has been curved to conform to the shape of the breast, and the scores of the scoring pattern 208 have splayed apart to conform to the curvature of the breast. The point 174″ is positioned proximate a bottom of the sternum.

Due to the symmetric shape of the manifold layer 204, the manifold layer 204 can be used to treat wounds in both the left breast (FIG. 10) and the right breast (FIG. 9) without requiring modification. As can best be seen in FIG. 10, the wound dressing 100 is sized so that a wound dressing 100 can simultaneously be used independently treat each breast.

### Wound Dressing

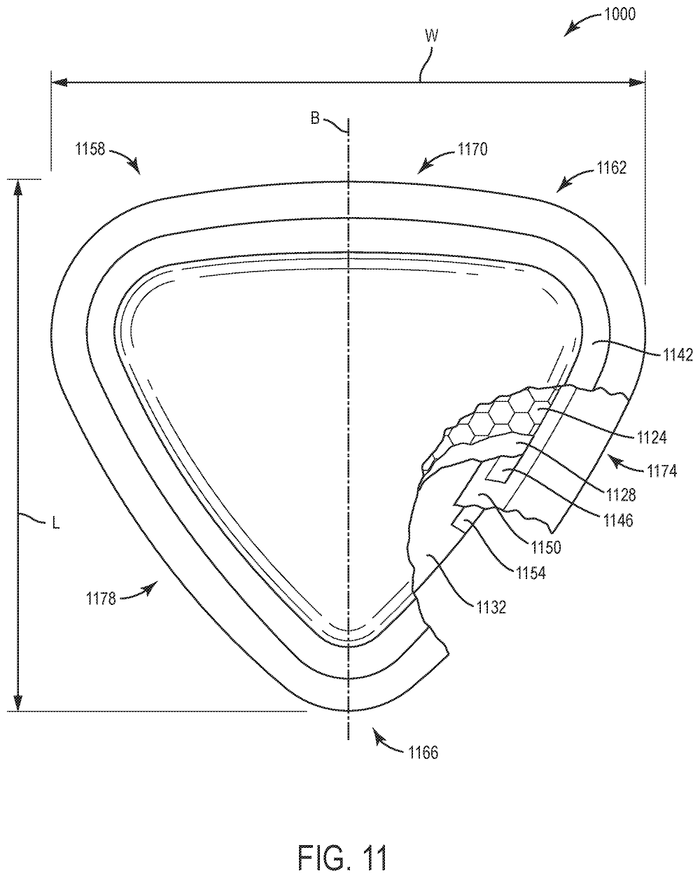

FIG. 11 illustrates a wound dressing 1000 having a single lobe according to an exemplary embodiment. The wound dressing 1000 is substantially similar to the wound dressing 100. Like parts between the wound dressings 100 and 1000 are indicated by preceding corresponding parts of the wound dressing 100 with the number 1. The wound dressing 1000 has a profile or a perimeter that is generally shaped like a guitar pick (e.g., generally triangular and having curved sides and corners). It is contemplated that the size of the wound dressing 1000 can range from 180 cm² to 1000 cm². More preferably, the size of the wound dressing 1000 can range from 370 cm² to 380 cm².

The wound dressing 1000 is shown to include a plurality of layers, including a drape layer 1120, a manifold layer 1124, a wound-interface layer 1128, a rigid support layer 1142, a first adhesive layer 1146, and a second adhesive layer 1150. In some embodiments, the wound dressing 1000 includes a removable cover sheet to cover the manifold layer 1124, the wound-interface layer 128, the second adhesive layer 1150, and/or the patient contacting layer 1154 before use.

### Manifold Layer

Referring to FIG. 11, the manifold layer 1124 includes a first surface and a second, wound-facing surface opposite the first surface. When the wound dressing 1000 is applied to a wound, the first surface faces away from the wound, whereas the second surface faces toward the wound. As described above, the manifold layer 1124 can be made from a porous and permeable foam-like material and, more particularly, a reticulated, open-cell polyurethane or polyether foam that allows good permeability of wound fluids while under a reduced pressure. One such foam material that has been used is the VAC® Granufoam™ material that is available from Kinetic Concepts, Inc. (KCl) of San Antonio, Tex. Any material or combination of materials might be used for the manifold layer 1124 provided that the manifold layer 1124 is operable to distribute the reduced pressure and provide a distributed compressive force along the wound site.

As shown in FIG. 11, the manifold layer 1124 is generally symmetrical and forms defines a single lobe that is generally shaped like a guitar pick (e.g., generally triangular and having curved sides and corners). The manifold layer 1124 includes a first curved corner 1158, a second curved corner 1162, and a third curved corner 1166. A first side 1170 extends between the first curved corner 1158 and the third curved corner 1166, a second side 1174 extends between the second curved corner 1162 and the third curved corner 1166, and a third side 1178 extends between the first curved corner 1158 and the second curved corner 1162. The manifold layer 1124 can have a width W ranging between 8 cm and 40 cm, and more preferably between 17 cm and 33 cm. The manifold layer 1124 can have a length L ranging between 7 cm and 35 cm, and more preferably between 14 cm and 30 cm. The manifold layer 1124 can have a thickness T ranging between 14 mm and 24 mm, and more preferably 19 mm. Each of the sides 1170, 1174, 1178 is generally convex and has a radius of curvature of approximately 42.3 cm. The first curved corner 1158 and the second curved corner 1162 are less curved than the third curved corner 1166. More specifically, the first curved corner 1158, the second curved corner 1162, and the third curved corner 1166 can each have a radius of curvature ranging between 3 cm and 10 cm. More preferably, the first curved corner 1158 and the second curved corner 1162 can each have a radius curvature of approximately 4.8 cm. More preferably, the third curved corner 1166 can have a radius of curvature of approximately 3.5 cm. In the illustrated embodiment, the wound dressing 1000 is generally symmetric about the axis B.

The manifold layer 1124 can have any of the scoring patterns 176, 184, 208, 216 described above. The manifold layer 1124 can be used in a wound therapy system similar to the wound therapy system described below and illustrated in FIG. 4.

The wound dressing 1000 can be positioned relative to a woman's torso in a manner similar to what is illustrated in FIGS. 9 and 10 for the wound dressing 100 and the manifold layer 204. The wound dressing 1000 is sized to cover the surface including the entire breast area, including the first incision 228, the second incision 232, and any incisions proximate the armpit as described above. More specifically, the third side 1178, extending between the first curved corner 1158 and the second curved corner 1162, is positioned proximate the patient's armpit and the third curved corner 1166 is oriented towards a bottom portion of the patient's sternum. Due to the symmetric shape of the wound dressing 1000, the wound dressing 1000 can be used to treat wounds in both the left breast and the right breast without requiring modification. For example, when the wound dressing 1000 is oriented to treat the right breast in a manner similar to what is illustrated with respect to the manifold layer 204 in FIG. 9, the second side 1174, which extends between the second curved corner 1162 and the third curved corner 1166, covers an upper portion of the breast area and the first side 1170, which extends between the first curved corner 1158 and the third curved corner 1166, covers a lower portion of the breast area. When the wound dressing 1000 is oriented to treat the left breast in a manner similar to what is illustrated with respect to the wound dressing 100 and the manifold layer 204 in FIG. 10, the first side 1170, which extends between the first curved corner 1158 and the third curved corner 1166, covers an upper portion of the breast area and the second side 1174, which extends between the second curved corner 1162 and the third curved corner 1166, covers a lower portion of the breast area.

### Wound Therapy System

Referring now to FIG. 4, a wound therapy system 236 is shown engaged with the surface 104 of a patient to treat a region proximate an incision 240, according to an exemplary embodiment. The incision 240 extends through the epidermis 244, or skin, and dermis 248, and reaches into a hypodermis 252, or subcutaneous tissue. The subcutaneous tissue 252 may include breast tissue, fatty tissue, or muscle. An undermined subcutaneous tissue site 252 is shown extending from the incision 240, and in the illustrated embodiment, shows a subcutaneous defect or void 256 that may be caused by surgical procedures, such as full or partial mastectomies, breast enhancements, and/or breast reductions. The incision 240 can be closed using any closing device such as sutures, staples, or an adhesive. In the illustrated embodiment, the incision 240 is closed using a suture 260.

The wound therapy system 236 further includes a removed fluid container 272 and a negative pressure source or pump 276 that are in fluid communication with the wound dressing 100 via the removed fluid conduit 268. The removed fluid container 272 can be configured to store a fluid removed from incision 240. Removed fluid can include, for example, wound exudate (e.g., bodily fluids), air, or any other type of fluid which can be removed from the incision 240 during wound treatment.

With continued reference to FIG. 4, the wound dressing 100 is positioned above the incision 240. The second adhesive layer 150 secures the wound dressing 100 to the patient. The wound-interface layer 128 contacts the surface 104 of the patient, and the manifold layer 204 is positioned above the wound-interface layer 128. The drape layer 120 extends over the manifold layer 204 to provide a fluid-tight seal of the wound dressing 100. The reduced-pressure interface 158 is engaged with an L-shaped connector 264 configured to engage a removed fluid conduit 268. In the illustrated embodiment, the removed fluid conduit 268 is a multi-lumen conduit. The removed fluid conduit 268 includes a first lumen 274 and a second lumen 278. The first lumen 274 is configured to apply negative pressure to the wound dressing 100 and to draw exudate into the removed fluid container 272. The second lumen 278 is configured for sensing the pressure of the wound dressing 100. One such wound therapy system 236 including a multi-lumen conduit is the SensaT.R.A.C.™ system that is available from Kinetic Concepts, Inc. (KCl) of San Antonio, Tex.

### Negative Pressure Wound Treatment Therapy

FIG. 10 illustrates a NPWT system engaged with the wound dressing 100. The NPWT system includes the pump 276 configured to apply a negative pressure to the wound site and the removed fluid container 272 to retain exudate from the wound site. In addition to providing NPWT, the negative pressure caused by the pump 276 provides support to the breast tissue. The compressive forces caused by the negative pressure hold any remaining breast tissue, reconstructive material, and/or implant material in position as the wound heals.

In embodiments in which both breasts are treated using the wound dressings 100, the wound dressings 100 may be connected to different pumps or the wound dressings 100 may be connected to the same pump 276 using a Y connector (not shown). In either configuration, the use of separate wound dressings on each breast allows the NPWT to be customized for each breast. For example, breasts having different sizes of wounds or wounds healing at different rates can have different NPWT requirements. Accordingly, the use of separate wound dressings 100 on each breast allows the treatment protocol for each wound to be customized.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

1.-7. (canceled)

8. A wound dressing for negative pressure wound therapy treatment of breast incisions including a manifold layer and a drape layer that is substantially impermeable to liquid and substantially permeable to vapor, the manifold layer comprising:
a first surface;
a second, wound-facing, surface; and
a plurality of scores formed in the first surface and extending towards the second surface, the plurality of scores defining a geometric scoring pattern, the manifold layer being bendable about the plurality of scores.

9. The wound dressing of claim 8, wherein the geometric pattern includes at least one of hexagons, quadrilaterals, or concentric curves.

10. The wound dressing of claim 8, wherein the plurality of scores is a first plurality of scores, and further comprising a second plurality of scores formed in the first surface and extending towards the second surface, the second plurality of scores including perforations that penetrate the second surface, the second plurality of scores defining a geometric scoring pattern.

11. The wound dressing of claim 10, wherein at least a portion of the manifold layer proximate the perforations is selectively removable to provide a window through the manifold layer.

12. The wound dressing of claim 11, wherein the manifold layer is configured to conform to a surface defined by a breast and the window of the manifold layer proximate the perforations is configured to be positioned proximate a nipple of the breast.

13. The wound dressing of claim 8, wherein the manifold layer is positionable in at least one of a first position in which the manifold layer conforms to a substantively two-dimensional shape with the scores substantially closed and a second position in which the manifold layer conforms to a substantively three-dimensional shape with at least some of the scores splayed apart.

14. The wound dressing of claim 8, further comprising a first curved side surface defining a first lobe, a second curved surface defining a second lobe, and a connecting portion between the first lobe and the second lobe, wherein the first lobe and the second lobe are convex or the connecting portion is concave.

15. The wound dressing of claim 14, wherein the one of the first lobe and the second lobe is configured to conform to a shape of an upper portion of a breast and the other of the first lobe and the second lobe is configured to conform to a shape of a lower portion of a breast.

16. The wound dressing of claim 8, wherein the manifold layer comprises a single lobe including a first curved corner having first radius of curvature, a second curved corner having a second radius of curvature, and a third curved corner having a third radius of curvature, and wherein the third radius of curvature is smaller than the first radius of curvature and the second radius of curvature.

17.-24. (canceled)
